(19) United States
(12) Patent Application Publication
(10) Pub. No.: US 2025/0127422 A1
(43) Pub. Date: Apr. 24, 2025

(54) MOTION STATE MONITORING SYSTEM, CONTROL METHOD, AND NON-TRANSITORY COMPUTER READABLE MEDIUM

(71) Applicant: TOYOTA JIDOSHA KABUSHIKI KAISHA, Toyota-shi (JP)

(72) Inventors: Makoto KOBAYASHI, Nisshin-shi (JP); Hiroto Ichikawa, Seto-shi (JP); Shuhao Chang, Kitanagoya-shi (JP)

(73) Assignee: TOYOTA JIDOSHA KABUSHIKI KAISHA, Toyota-shi (JP)

(21) Appl. No.: 18/824,217

(22) Filed: Sep. 4, 2024

(30) Foreign Application Priority Data
Oct. 24, 2023 (JP) 2023-182858

(51) Int. Cl. A61B 5/11; A61B 5/00

(52) U.S. Cl. CPC A61B 5/11; A61B 5/7435

(57) ABSTRACT

It is specified which sensor icons displayed on a display unit a plurality of sensors used in a motion state monitoring apparatus correspond to. A motion state monitoring system according to this embodiment is a motion state monitoring system including a plurality of sensors corresponding to a plurality of respective body parts of a body of a subject and a motion state monitoring apparatus configured to monitor motions of the subject in accordance with results of detection from the plurality of sensors, in which the motion state monitoring apparatus includes a display unit configured to display a plurality of sensor icons corresponding to the plurality of respective sensors; and a display control unit configured to change a display aspect of the corresponding sensor icon in accordance with an input from one of the plurality of sensors.

### CROSS REFERENCE TO RELATED APPLICATIONS

This application is based upon and claims the benefit of priority from Japanese patent application No. 2023-182858, filed on Oct. 24, 2023, the disclosure of which is incorporated herein in its entirety by reference.

### BACKGROUND

The present disclosure relates to a motion state monitoring system, a control method, and a program.

Japanese Unexamined Patent Application Publication No. 2022-34449 discloses a motion state monitoring system configured to monitor a motion state of a subject based on results of detection performed in a plurality of sensors attached to a plurality of respective body parts of a body of a subject.

### SUMMARY

In a motion state monitoring system as disclosed in related art, positions to which a plurality of sensors are attached may be displayed on a display unit of a motion state monitoring apparatus by icons or the like. In this case, there is a problem that it is impossible to grasp which icons the respective sensors correspond to.

The present disclosure has been made in view of the aforementioned circumstances and an object thereof is to provide a motion state monitoring system, a control method, and a program capable of specifying which sensor icons displayed on a display unit a plurality of sensors used in a motion state monitoring apparatus correspond to.

A motion state monitoring system according to the present disclosure is a motion state monitoring system including a plurality of sensors corresponding to a plurality of respective body parts of a body of a subject and a motion state monitoring apparatus configured to monitor motions of the subject in accordance with results of detection from the plurality of sensors, in which the motion state monitoring apparatus includes: a display unit configured to display a plurality of sensor icons corresponding to the plurality of respective sensors; and a display control unit configured to change a display aspect of the corresponding sensor icon in accordance with an input from one of the plurality of sensors.

With the above motion state monitoring system, it becomes possible to change a display aspect of a sensor icon in accordance with an input from a sensor. It becomes therefore possible for a user to easily recognize the sensor icon corresponding to the sensor by only checking a display unit.

Further, this motion state monitoring system may perform calculation processing using a learned model generated by machine learning that uses previous results of detection in sensors. This motion state monitoring system performs calculation processing using a learned model, whereby it is possible to calculate whether or not the motion state of the motion to be monitored of the subject is fine more accurately.

The display control unit may change a display aspect in such a way that the corresponding sensor icon performs a motion in accordance with the motion of one of the plurality of sensors. Further, the display control unit may rotate the corresponding sensor icon in accordance with a rotation operation of the sensor with a predetermined axis as a rotation axis.

The sensor may include an acceleration sensor. The display control unit may change a display aspect based on a tap input on the sensor collected by the acceleration sensor.

The sensor may include a switch. The display control unit may change a display aspect based on a state of the switch being pressed.

The display unit may display a diagram of a human body showing body parts to which sensors are to be attached. The sensor icon may be displayed on the body parts to which sensors are to be attached.

A method for controlling a motion state monitoring system according to the present disclosure is a method for controlling a motion state monitoring system including a plurality of sensors corresponding to a plurality of respective body parts of a body of a subject and a motion state monitoring apparatus configured to monitor motions of the subject in accordance with results of detection from the plurality of sensors, in which the motion state monitoring apparatus executes: processing for displaying a plurality of sensor icons that are made to correspond to the plurality of respective sensors; and processing for changing a display aspect of the corresponding sensor icon in accordance with an input from one of the plurality of sensors.

In the method for controlling the motion state monitoring system, it becomes possible to change a display aspect of a sensor icon in accordance with an input from a sensor. It becomes therefore possible for a user to easily recognize the sensor icon corresponding to the sensor by only checking a display unit.

A program according to the present disclosure is a program for controlling a motion state monitoring system including a plurality of sensors corresponding to a plurality of respective body parts of a body of a subject and a motion state monitoring apparatus configured to monitor motions of the subject in accordance with results of detection from the plurality of sensors, the program causing the motion state monitoring apparatus to execute: processing for displaying a plurality of sensor icons that are made to correspond to the plurality of respective sensors; and processing for changing a display aspect of the corresponding sensor icon in accordance with an input from one of the plurality of sensors.

According to this program, it becomes possible to change a display aspect of a sensor icon in accordance with an input from a sensor. It becomes therefore possible for a user to easily recognize the sensor icon corresponding to the sensor by only checking a display unit.

According to the present disclosure, it becomes possible to provide a motion state monitoring system, a control method, and a program capable of specifying which sensor icons displayed on a display unit a plurality of sensors used in a motion state monitoring apparatus correspond to.

The above and other objects, features and advantages of the present disclosure will become more fully understood from the detailed description given hereinbelow and the accompanying drawings.

### BRIEF DESCRIPTION OF DRAWINGS

FIG. 1 is a block diagram showing a configuration example of a motion state monitoring system according to an embodiment;

FIG. 2 is a diagram showing one example of body parts of a subject to which measuring instruments are to be attached;

FIG. 3 is a diagram showing a configuration example of the measuring instrument provided in the motion state monitoring system according to the embodiment;

FIG. 4 is a diagram showing one example of how to attach the measuring instrument shown in FIG. 3 to the subject;

FIG. 5 is a diagram showing one example of a display screen;

FIG. 6 is a diagram showing one example of the display screen;

FIG. 7 is a diagram showing one example of the display screen;

FIG. 8 is a diagram showing one example of the display screen; and

FIG. 9 is a flowchart showing an operation of a motion state monitoring apparatus provided in the motion state monitoring system according to the embodiment.

### DESCRIPTION OF EMBODIMENTS

Embodiments of the present disclosure will now be described with reference to the drawings. However, the claimed disclosure is not limited to the following embodiments. Moreover, not all of the configurations described in the embodiments are essential as means for solving the problem. For the sake of clarity, the following descriptions and drawings have been omitted and simplified as appropriate. In each drawing, the same elements have the same reference signs, and repeated descriptions have been omitted as appropriate.

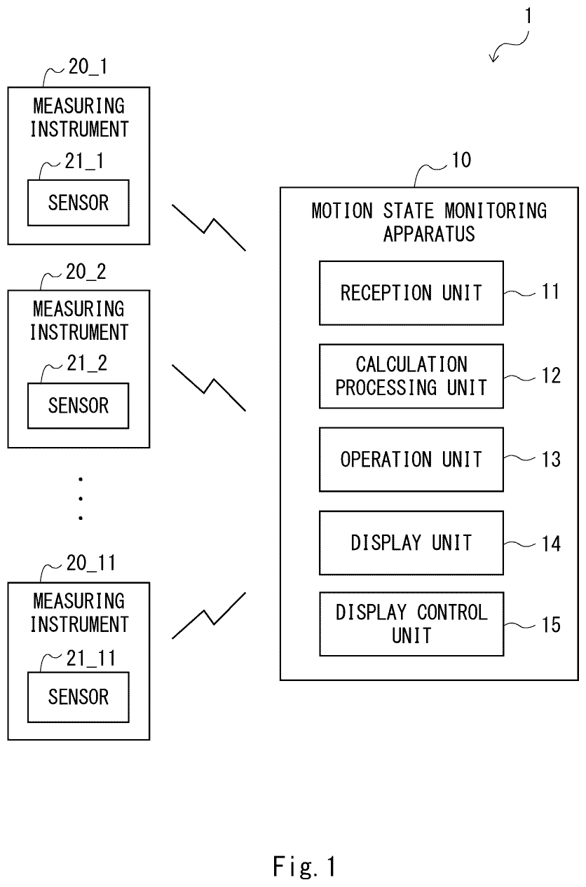

FIG. 1 is a block diagram showing a configuration example of a motion state monitoring system 1 according to an embodiment. The motion state monitoring system 1 is a system that monitors a motion state of a subject. With this result of the monitoring, a user such as an assistant, for example, is able to perform support for making a motion of a subject close to a desired motion. The details will be described below.

As shown in FIG. 1, the motion state monitoring system 1 includes a plurality of measuring instruments 20 and a motion state monitoring apparatus 10. The motion state monitoring apparatus 10 itself may be referred to as a motion state monitoring system. The motion state monitoring apparatus 10 and 15 the plurality of measuring instruments 20 are configured to be able to communicate with each other via a wired or wireless network. In this embodiment, an example in which 11 measuring instruments 20 are provided will be described. In the following, 11 measuring instruments 20 are also referred to as measuring instruments 20_1 to 20_11, respectively, in order to 20 distinguish them from each other.

The measuring instruments 20_1 to 20_11 are attached to respective body parts p1 to p11 from which motions are to be detected among various body parts of the body of a subject P, and detect the motions of the respective body 25 parts p1 to p11 using motion sensors (hereinafter simply referred to as sensors) 21_1 to 21_11 formed of a gyro sensor, an acceleration sensor or the like. Hereinafter, the sensors 21_1 to 21_11 will also be collectively referred to as a sensor(s) 21. Note that the sensors 21_1 to 21_11 are respectively made to correspond to the body parts p1 to p11 by correspondence processing performed 30 between the sensors 21_1 to 21_11 and the motion state monitoring apparatus 10 that will be described below.

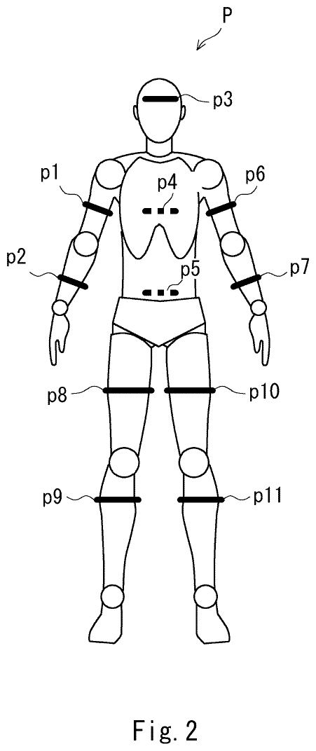

FIG. 2 is a diagram showing one example of the body parts to which the measuring instruments 20_1 to 20_11 are to be attached. In the example shown in FIG. 2, the body parts p1 to p11 to which the measuring instruments 20_1 to 20_11 are to be attached are a right upper arm, a right forearm, a head, a back (a trunk), a waist (a pelvis), a left upper arm, a left forearm, a right thigh, a right lower leg, a left thigh, and a left lower leg, respectively. It is assumed, in this example, that the back and the waist are positioned on the back side of the subject P. Note that not all the measuring instruments 20_1 to 20_11 need to be attached to the body of the subject P. It is sufficient that, among the measuring instruments 20_1 to 20_11, only measuring instruments that are necessary to measure the motions to be monitored (including motions of body parts) that the user wants to monitor be attached to the body of the subject P.

### Configuration Example of Measuring Instruments 20_1 to 20_11

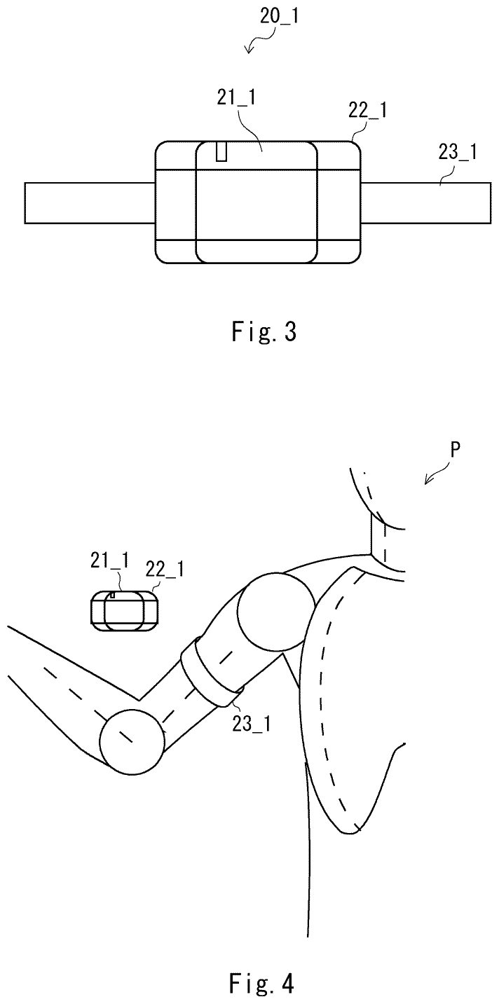

FIG. 3 is a diagram showing a configuration example of the measuring instrument 20_1. Since the configuration of the measuring instruments 20_2 to 20_11 is the same as that of the measuring instrument 20_1, the descriptions thereof will be omitted.

As shown in FIG. 3, the measuring instrument 20_1 includes a sensor 21_1, an attachment pad 22_1, and a belt 23_1. The belt 23_1 is configured so that it can be wound around the body part of the subject P from which a motion is to be detected. The sensor 21_1 is integrated with, for example, the attachment pad 22_1. Further, the attachment pad 22_1 with which the sensor 21_1 is integrated is configured so that it can be attached to or detached from the belt 23_1.

FIG. 4 is a diagram showing an example of how to attach the measuring instrument 20_1 to the subject P. In the example shown in FIG. 4, the belt 23_1 is wound around the right upper arm, which is one of the body parts of the subject P from which motions are to be detected. The sensor 21_1 is attached to the belt 23_1 with the attachment pad 22_1 interposed therebetween after correspondence processing, calibration, and the like have been completed.

### Configuration Example of Motion State Monitoring Apparatus 10

The motion state monitoring apparatus 10 is an apparatus that outputs a result of a calculation indicating a motion state of the subject P based on results of detection (sensing values) performed by the sensors 21_1 to 21_11. The motion state monitoring apparatus 10 may be, for example, a Personal Computer (PC), a mobile phone terminal, a smartphone, a tablet terminal, or the like. The motion state monitoring apparatus 10 is configured to be able to communicate with the sensors 21_1 to 21_11 via a network (not shown).

The motion state monitoring apparatus 10 includes a communication unit 11, a calculation processing unit 12, an operation unit 13, a display unit 14, and a display control unit 15. The communication unit 11 is a communication interface with a network. The motion state monitoring apparatus 10 is able to receive identification information of the sensors 21_1 to 21_11 and a result of detection via the communication unit 11.

Note that the communication unit 11 may establish the connection of short-range wireless communication and perform communication. Various kinds of standards such as Bluetooth (registered trademark), Bluetooth Low Energy (BLE), or Ultra-Wide Band (UWB) can be applied to the short-range wireless communication. For example, the communication unit 11 can receive identification information from each of the plurality of sensors 21_1 to 21_11 located within a predetermined distance by short-range wireless communication.

It is assumed here that the communication unit 11 performs data communication conforming to Bluetooth (registered trademark) standard as the short-range wireless communication. The motion state monitoring apparatus 10 and a sensor 21 that is present within a communication range are paired with each other by exchanging identification information such as a Bluetooth address and mutually authenticating with each other, whereby the motion state monitoring apparatus 10 is connected to the sensor 21. Once the pairing is completed, necessary information is stored in each other's equipment. In the following processing, when the sensor 21 is located within a predetermined distance from the motion state monitoring apparatus 10, they are connected to each other without performing pairing.

The calculation processing unit 12 performs calculation processing based on a result of detection performed by each of the sensors 21_1 to 21_11 to generate a result of the calculation indicating the motion state of the motion to be monitored of the subject P. The motions to be monitored include, for example, motions such as bending and stretching of the right shoulder, adduction and abduction of the right shoulder, internal and external rotation of the right shoulder, bending and stretching of the right elbow, pronation and supination of the right forearm, bending and stretching of the head, rotation of the head, bending and stretching of the chest and the waist, rotation of the chest and the waist, lateral bending of the chest and the waist, bending and stretching of the left shoulder, adduction and abduction of the left shoulder, internal and external rotation of the left shoulder, bending and stretching of the left elbow, pronation and supination of the left forearm. Further, the motions to be monitored include the motion of the body part to which a sensor is attached. For example, the motions to be monitored include angles of joints of the body of the subject P measured based on results of detection performed by the plurality of sensors or angles of joints in any coordinate system measured based on a result of detection performed by any one of the sensors. In the following, generation of the result of the calculation indicating the motion state of the motions to be monitored is also referred to as measurement of the motions to be monitored.

For example, the calculation processing unit 12 performs calculation processing based on a result of detection in each of the sensor 21_1 attached to the right upper arm (the body part p1) of the subject P and the sensor 21_2 attached to the right forearm (the body part p2) of the subject P, of the sensors 21_1 to 21_11, to generate the result of the calculation indicating the motion state of the bending and stretching motion of the right elbow of the subject P.

Alternatively, the calculation processing unit 12 performs calculation processing based on a result of detection in each of the sensor 21_5 attached to the waist (the body part p5) of the subject P and the sensor 21_8 attached to the right thigh (the body part p8) of the subject P, of the sensors 21_1 to 21_11, to generate the result of the calculation indicating the motion state of the lateral bending of the waist on the right side of the subject P.

Note that the calculation processing unit 12 may perform calculation processing using a learned model generated by machine learning that uses previous results of detection in sensors. The calculation processing unit 12 may accurately calculate whether or not the motion state of the motion to be monitored of the subject P is fine by performing calculation processing using the above learned model. The calculation processing unit 12 transmits the result of the calculation to the display control unit 15.

The operation unit 13 may include an input apparatus such as a mouse or a keyboard. The operation unit 13 may be a touch panel in which a display apparatus and an input apparatus are integrated with each other. For example, the user enters information regarding the subject, a result of monitoring to be displayed on the display unit 14, or the like into the display unit 14 by operating a mouse, a keyboard or the like of the operation unit 13 or operating a touch panel of the operation unit 13 with a touch pen or his/her finger.

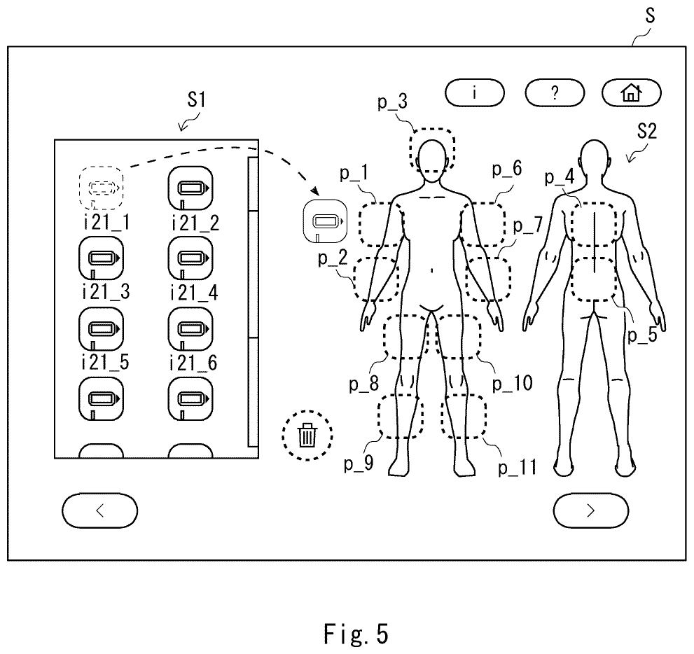

The display unit 14 is a display apparatus that displays a predetermined image on a screen. The display unit 14 displays a plurality of sensor icons corresponding to a plurality of sensors 21_1 to 21_11, respectively. FIG. 5 shows one example of a display screen S displayed on the display unit 14. The area of the display screen S which displays the sensor icon is referred to as a sensor icon display area S1.

The display unit 14 may display a diagram of a human body showing body parts to which the sensors 21_1 to 21_11 are to be attached. The display screen including the sensor icon display area S1 includes a human body schematic diagram S2 showing body parts to which sensors are to be attached. The display screen S shown in FIG. 5 is displayed when, for example, processing for making one of the plurality of sensors 21 correspond to one of the body parts p of the subject P to which the sensors are to be attached in one-to-one is performed.

In the example shown in FIG. 5, the human body schematic diagram S2 displays the front side and the back side separately. The body parts p1 to p11 of the subject P to which sensors are to be attached in FIG. 2 respectively correspond to body parts p_1 to p_11 of the human body schematic diagram S2 in FIG. 5. Hereinafter, the body parts p_1 to p_11 on the human body schematic diagram S2 are also referred to as a body part(s) p_. Hereinafter, as necessary, p_1 to p_11 of the human body schematic diagram S2 are respectively referred to as a right upper arm, a right forearm, a head, a back (a trunk), a waist (a pelvis), a left upper arm, a left forearm, a right thigh, a right lower leg, a left thigh, and a left lower leg. Further, the display unit 14 is able to display the result of the calculation based on the result of the detection performed by each of one or more sensors in a form of a graph, for example.

Further, the display unit 14 displays, when it receives an operation performed by a user, an input screen of information on the subject, a screen for selecting a result of the monitoring to be displayed on the display unit 14, or a result of the monitoring generated after the motion state of the subject is monitored. While the motion state monitoring apparatus 10 includes the operation unit 13 and the display unit 14 in the example shown in FIG. 1, the operation unit 13 and the display unit 14 may be formed as separate operation terminals.

The display control unit 15 causes the display unit 14 to display, based on the received identification information of the sensors 21_1 to 21_11, sensor icons that are different for each sensor. That is, the display unit 14 displays a list of sensor icons corresponding to the respective available sensors that have not been made to correspond to any body part to which the sensor is to be attached. These plurality of sensor icons displayed on the icon display unit S1 are selected by a user in order to make the sensor 21 correspond to the body part p of the subject to which the sensor is to be attached.

For example, the user operates the operation unit 13 to move a sensor icon i21_1 displayed in the sensor icon display area S1 to the right upper arm p_1 on the human body schematic diagram S2, as shown by a dotted arrow in FIG. 5. Specifically, the user drags and drops the sensor icon i21_1 displayed in the sensor icon display area S1 onto the right upper arm p_1 of the human body schematic diagram S2 by a mouse operation, a touch operation or the like.

Accordingly, the setting operation on the sensor icon by the user is received, and the sensor corresponding to the sensor icon is made to correspond to the body part of the subject to which the sensor is to be attached in accordance with the setting operation. This correspondence processing is performed by pairing processing performed between the motion state monitoring apparatus 10 and the sensor 21 in advance, and making identification information of the body part to which the sensor is to be attached correspond to identification information of the sensor 21 on the application of the motion state monitoring apparatus 10.

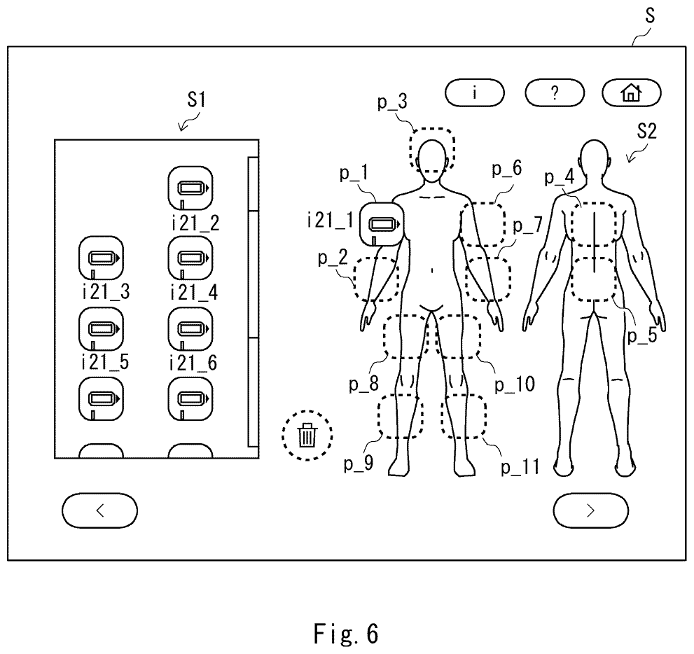

After the correspondence processing is performed, as shown in FIG. 6, the sensor icon i21_1 disappears from the icon display unit S1 and is displayed on the body part p_1 of the human body schematic diagram S2. In this manner, by a simple operation of moving a desired sensor icon to one of the body parts p_1 to p_11 on the human body schematic diagram S2, the sensor corresponding to the sensor icon can be made to correspond to the body parts p1 to p11 of the subject P. Note that the sensors 21_1 to 21_11 may be made to correspond to the body parts p1 to p11 in series, respectively. That is, the sensors 21_1 to 21_11 may be made to correspond exclusively to the body parts p1 to p11, respectively.

The plurality of sensors 21 may each include a light-emitting unit capable of changing a luminescence color thereof. The light-emitting unit may be, for example, a full-color LED. As one example, the light-emitting unit may have a configuration in which a red LED chip, a green LED chip, and a blue LED chip are sealed by resin having optical transparency. The light-emitting unit can provide a wide variety of luminescence colors by controlling brightness of three kinds of LED chips.

The motion state monitoring apparatus 10 may further include a color control unit configured to execute processing for making the display colors of the plurality of sensor icons the same as the luminescence colors of the light-emitting units of the plurality of respective sensors. The user can select a sensor of the luminescence color which is the same as the display color of the sensor icon, check the human body schematic diagram S2, and actually attach this sensor to the body part of the subject P to which the sensor is to be attached. Accordingly, it becomes possible to prevent a wrong sensor from being attached.

The display control unit 15 changes, in accordance with the input from one of the sensors 21_1 to 21_11, the display aspect of the corresponding sensor icons i21_1 to i21_11 displayed on the display unit 14. For example, the display control unit 15 is able to temporally change the target sensor icon displayed on the screen of the display unit 14 in association with the motion of the sensor 21.

The sensors 21 attached to the subject P generally move in a three-dimensional way. For example, the sensor 21 may include a gyro sensor and is able to detect each of an angular velocity about an X axis, that of a Y axis, and that of a Z axis that are perpendicular to each other. The display control unit 15 is able to rotate the sensor icon using, for example, a result of detection from the sensor 21 regarding any one of the X axis, the Y axis, and the Z axis.

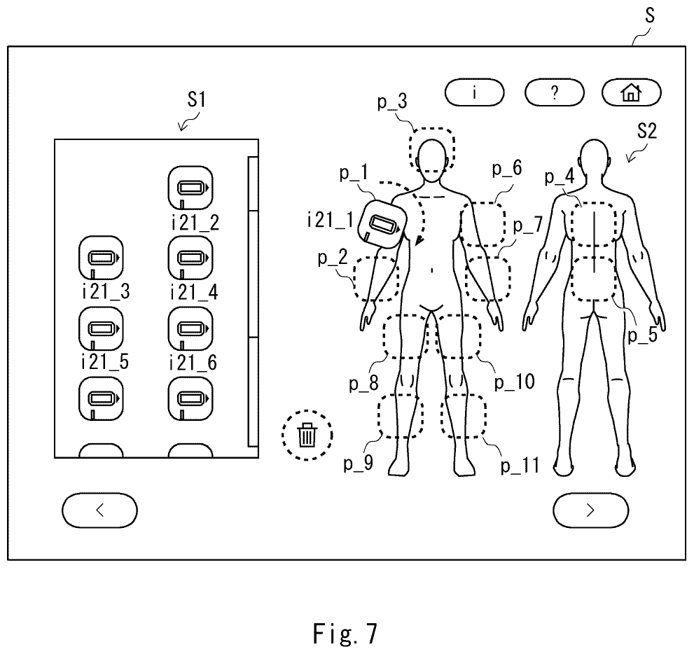

The display control unit 15 is able to rotate the target sensor icon i21_1 in accordance with a rotation operation of the sensor 21_1 with a predetermined axis as a rotation axis. As one example, as shown in FIG. 7, the display control unit 15 is able to cause the display unit 14 to display an image showing the rotation of the target sensor icon i21_1 with an axis passing through the center of mass of the target sensor icon i21_1 as a rotation axis, as shown by a dotted arrow in FIG. 7.

As described above, by changing the display aspect of the sensor icon in accordance with the motion of the sensor 21, the user is able to easily recognize which sensor icon displayed on the display unit 14 the sensor 21 corresponds to.

The change in the display aspect of the sensor icon is not limited to rotation of the sensor icon. For example, the display control unit 15 may change the color and the size of the sensor icon in accordance with an input from a sensor. Further, the sensor icons may flash or may be vibrated.

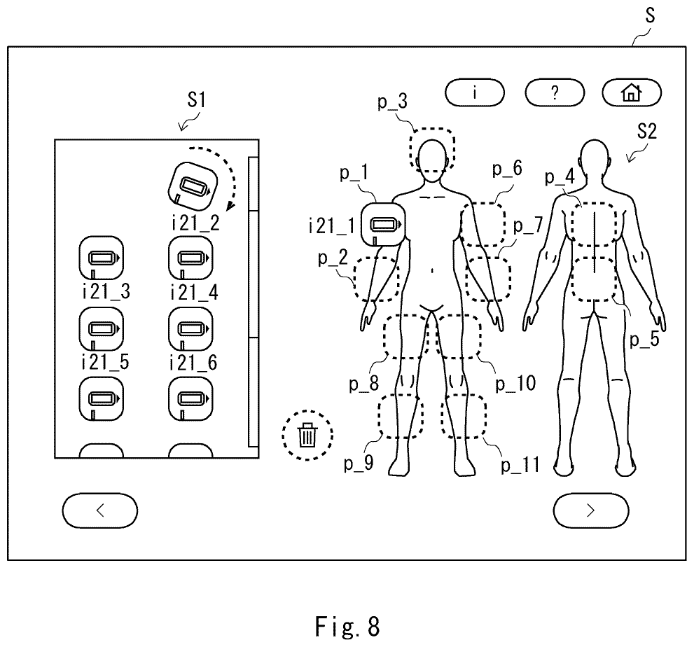

The processing for changing the display aspect of the sensor icon in accordance with the motion of the sensor 21 by the display control unit 15 may be performed before the sensors are attached to the respective body parts of the subject P or may be performed after the sensors are attached to the respective body parts of the subject P. When, for example, the sensor 21_2 is rotated before the sensor 21_2 is attached to the body part p2 of the subject P, the sensor icon i21_2 displayed on the icon display unit S1 may be rotated as shown in FIG. 8.

Even in a case where the sensor 21 is attached to the subject P, it is possible that the sensor 21 may not be seen when, for example, it is attached under the clothing of the subject P or the sensor 21 is hidden behind an attachment member such as the attachment pad 22_1 or the belt 23_1. In this case, after the sensor 21 is attached to the subject P, processing for changing the display aspect of the sensor icon in accordance with the motion of the sensor 21 is performed. It is therefore possible to easily specify the sensor icon that corresponds to the sensor 21 by only slightly moving the sensor 21.

Note that the display control unit 15 is able to temporally change the target sensor icon displayed on the screen of the display unit 14 in accordance with, not only the motion of the sensor 21, but also a physical input on the sensor 21. For example, the sensor 21 may include an acceleration sensor. The display control unit 15 is able to change the display aspect based on a tap input on the sensor 21 collected by the acceleration sensor. For example, the user performs a tap input into the sensor 21 using his/her finger or the like at an intensity that exceeds a threshold intensity, whereby the display control unit 15 may change the display aspect of the corresponding sensor icon.

Further, the sensor 21 may include a switch for changing the display aspect of the sensor icon. The display control unit 15 is able to change the display aspect based on a state of the switch being pressed. For example, the user presses a switch button a plurality of times within a predetermined period of time or presses the switch button for a time exceeding a threshold time, whereby the display control unit 15 can change the display aspect of the corresponding sensor icon.

Further, the display aspect of the sensor icon may be changed based on a change in an environment near the sensor 21. The sensor 21 may include, for example, a detector that detects a pressure, a temperature, illumination, or sound vibration of an environment near the sensor 21. For example, the user may cause a change in the pressure or the temperature near the sensor 21 by covering the sensor 21 with his/her hand. Further, the user may change the brightness near the sensor 21 by putting, for example, the sensor 21 into the attachment pad 22_1. The display control unit 15 may change the display aspect of the corresponding sensor icon in accordance with the change in the pressure, the temperature, or the illumination near the sensor 21.

Further, the user may generate a sound by, for example, snapping his/her finger near the sensor 21. The display control unit 15 may change the display aspect of the corresponding sensor icon when the sensor 21 has detected a sound whose volume level exceeds a threshold. Further, a change in the radio wave intensity between the sensor 21 and the motion state monitoring apparatus 10 that are paired with each other may be detected, and a display aspect of the sensor icon may be changed.

Further, the display control unit 15 causes the display unit 14 to display the information received from the calculation processing unit 12 (the result of the calculation) in a form of a graph or the like. Accordingly, the user can know the motion state of the motion to be monitored of the subject P, and the user can use this motion state to, for example, assist the subject P.

### Motions of Motion State Monitoring Apparatus 10

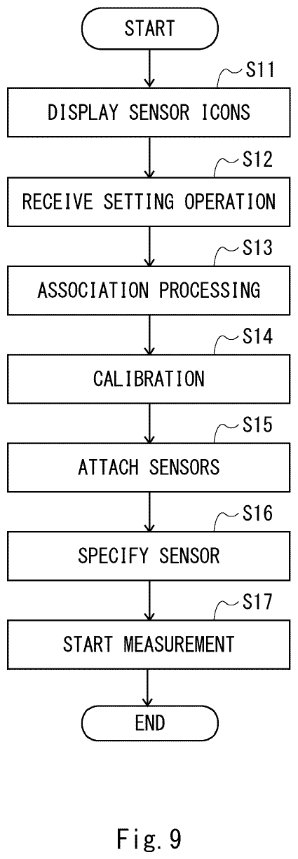

Referring next to FIG. 9, motions of the motion state monitoring apparatus 10 will be described. FIG. 9 is a flowchart showing the motions of the motion state monitoring apparatus 10. It is assumed that the bending and stretching motion of the right elbow of the subject P is monitored by the motion state monitoring apparatus 10. That is, it is assumed that sensors 21_1 and 21_2 are respectively made to correspond to the right upper arm (the body part p1) and the right forearm (the body part p2) of the subject P.

As shown in FIG. 9, first, the motion state monitoring apparatus 10 causes the display unit 14 to display sensor icon i21_1 to i21_11 corresponding to the plurality of respective sensors that are each within a predetermined distance (S11). At this time, not all the sensor icons corresponding to all the sensors 21_1 to 21_11 are displayed in the motion state monitoring apparatus 10 and some sensor icons may not be displayed depending on the distance from the motion state monitoring apparatus 10.

It is assumed here, as one example, that all the sensors 21_1 to 21_11 are each located within a predetermined distance from the motion state monitoring apparatus 10. It is therefore assumed that all the sensor icons i21_1 to i21_1 respectively corresponding to all the sensors 21_1 to 21_11 are displayed in the motion state monitoring apparatus 10.

The display unit 14 displays a display screen including the sensor icon display area S1 which displays sensor icons corresponding to the sensors distances of which from the motion state monitoring apparatus 10 are each within a predetermined distance and the human body schematic diagram S2 in which the body parts to which the sensors are to be attached are displayed, as shown in FIG. 5.

When the user specifies the motions to be monitored of the subject P, the display unit 14 may display the body parts to which sensors used to measure the specified motions to be monitored are to be attached. The display unit 14 of the motion state monitoring apparatus 10 may highlight the right upper arm p_1 and the right forearm p2 in the human body schematic diagram S2 to indicate that the right upper arm p_1 and the right forearm p2 have a display aspect (color, flashing, shading, or the like) different from those of the other body parts p3 to p11. That is, in the human body schematic diagram S2 shown in FIG. 5, the right upper arm p_1 and the right forearm p2 may have a display aspect different from those of the other body parts p3 to p11.

Then, the user drags and drops, for example, one sensor icon i21_1 onto the right upper arm p_1 in the human body schematic diagram S2 from the sensor icons i21_1 to i21_11 displayed in the sensor icon display area S1. Accordingly, the motion state monitoring system 1 receives the setting operation (S12).

After that, the motion state monitoring apparatus 10 makes, in accordance with the setting operation, identification information of the sensor 21_1 corresponding to the sensor icon i21_1 correspond to identification information of the right upper arm (the body part p1) of the subject P1 to which the sensor is to be attached. Accordingly, the processing for making the sensor 21_1 correspond to the right upper arm (the body part p1) of the subject P1 is performed (S13). Drag and drop of the sensor icon i21_2 and processing for making the sensor 21_2 correspond to the right forearm (the body part p2) of the subject P1 are performed for another body part to which the sensor is to be attached (right forearm (the body part p2)) as well. That is, S12 and S13 in FIG. 7 may be repeated for the number of body parts of the subject P1 to which sensors are to be attached.

After the correspondence of the sensor 21_1 with the body part p1 to which the sensor 21_1 is to be attached and the correspondence of the sensor 21_2 with the body part p2 to which the sensor 21_2 is to be attached are completed, calibration of the sensors 21_1 and 21_2 used to measure the motions to be monitored is performed (S14). The calibration indicates, for example, processing for measuring an output value (error component) in a stationary state of the sensor used to measure the motion to be monitored and subtracting an error component from the measured value. In this example, calibration of at least the sensors 21_1 and 21_2 is performed. However, the calibration may be performed on not only the sensor used to measure the motion to be monitored but also all the sensors 21_1 to 21_11 at a timing before, for example, the processing for displaying the sensor icon corresponding to the sensor that has already been subjected to pairing.

Then, the sensors 21_1 and 21_2 are attached to the subject P1 (S15). Then, after the measuring instruments 20_1 and 20_2 into which the sensors 21_1 and 21_2 are incorporated are actually attached to the subject P, the sensor 21 can be specified (S16). The sensor 21 can be specified by moving the sensor 21 and changing the display aspect of the sensor icon. As one example, by rotating the sensor 21_1, the sensor icon i21_1 is rotated, as shown in FIG. 7. Accordingly, even in a case where the sensors 21_1 and 21_2 after being attached cannot be seen, it becomes possible to specify the corresponding sensor icons displayed on the display unit 14.

After that, the motions to be monitored are measured based on the result of the detection in each of the sensors 21_1 and 21_2 (S17). The result of the calculation indicating the motion state of “bending and stretching of the right elbow” can be calculated from the difference between the result of the detection performed in the sensor 21_1 attached to the right upper arm (the body part p1) of the subject P1 and the result of the detection performed in the sensor 21_2 attached to the right forearm (the body part p2). The motion state monitoring apparatus 10 generates the result of the calculation indicating the motion state of “bending and stretching of the right elbow” based on the result of the detection performed by each of the sensors 21_1 and 21_2. The display unit 14 displays the details of the result of the measurement (e.g., a result of measurement shown in a form of a graph).

As described above, in the motion state monitoring system 1 according to this embodiment, by only slightly moving the sensor 21, it becomes possible to specify which one of the sensor icons displayed on the display unit 14 each of a plurality of sensors used in a motion state monitoring apparatus corresponds to. Accordingly, usability of the motion state monitoring system according to this embodiment can be improved.

Further, the present disclosure may implement a part or all of the processing in the motion state monitoring system 1 by causing a Central Processing Unit (CPU) to execute a computer program.

The aforementioned program includes instructions (or software codes) that, when loaded into a computer, cause the computer to perform one or more of the functions described in the embodiments. The program may be stored in a non-transitory computer readable medium or a tangible storage medium. By way of example, and not a limitation, computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (registered trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

The program can be stored and provided to a computer using any type of non-transitory computer readable media. Non-transitory computer readable media include any type of tangible storage media. Examples of non-transitory computer readable media include magnetic storage media (such as flexible disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A motion state monitoring system comprising a plurality of sensors corresponding to a plurality of respective body parts of a body of a subject and a motion state monitoring apparatus configured to monitor motions of the subject in accordance with results of detection from the plurality of sensors, wherein
the motion state monitoring apparatus comprises:
a display unit configured to display a plurality of sensor icons corresponding to the plurality of respective sensors; and
a display control unit configured to change a display aspect of the corresponding sensor icon in accordance with an input from one of the plurality of sensors.

2. The motion state monitoring system according to claim 1, wherein the display control unit changes a display aspect in such a way that the corresponding sensor icon performs a motion in accordance with the motion of one of the plurality of sensors.

3. The motion state monitoring system according to claim 2, wherein the display control unit rotates the corresponding sensor icon in accordance with a rotation operation of the sensor with a predetermined axis as a rotation axis.

4. The motion state monitoring system according to claim 1, wherein
the sensor includes an acceleration sensor, and
the display control unit changes a display aspect based on a tap input on the sensor collected by the acceleration sensor.

5. The motion state monitoring system according to claim 1, wherein
the sensor includes a switch, and
the display control unit changes a display aspect based on a state of the switch being pressed.

6. The motion state monitoring system according to claim 1, wherein
the display unit displays a diagram of a human body showing body parts to which the sensors are to be attached, and
the sensor icon is displayed on the body parts to which the sensors are to be attached.

7. A method for controlling a motion state monitoring system comprising a plurality of sensors corresponding to a plurality of respective body parts of a body of a subject and a motion state monitoring apparatus configured to monitor motions of the subject in accordance with results of detection from the plurality of sensors, in which
the motion state monitoring apparatus executes:
processing for displaying a plurality of sensor icons that are made to correspond to the plurality of respective sensors; and
processing for changing a display aspect of the corresponding sensor icon in accordance with an input from one of the plurality of sensors.

8. A non-transitory computer readable medium storing a program for controlling a motion state monitoring system comprising a plurality of sensors corresponding to a plurality of respective body parts of a body of a subject and a motion state monitoring apparatus configured to monitor motions of the subject in accordance with results of detection from the plurality of sensors, the program causing the motion state monitoring apparatus to execute:
processing for displaying a plurality of sensor icons that are made to correspond to the plurality of respective sensors; and
processing for changing a display aspect of the corresponding sensor icon in accordance with an input from one of the plurality of sensors.
